(19) United States
(12) Patent Application Publication
(10) Pub. No.: US 2019/0050863 A1
(43) Pub. Date: Feb. 14, 2019

(54) ELECTRONIC SYSTEM AND METHOD FOR MAKING GROUP PAYMENTS

(71) Applicant: Mastercard International Incorporated, Purchase, NY (US)

(72) Inventor: Rahul Agrawal, Pune (IN)

(73) Assignee: Mastercard International Incorporated, Purchase, NY (US)

(21) Appl. No.: 16/018,858

(22) Filed: Jun. 26, 2018

(30) Foreign Application Priority Data
Aug. 8, 2017 (SG) 10201706487S

(51) Int. Cl. G06Q 20/40; G06Q 20/36

(52) U.S. Cl. CPC G06Q 20/40145; A61B 5/0245; G06Q 20/405; G06Q 20/36

(57) ABSTRACT

An electronic system and method is described for authorising a group payment from a group account. The system comprises a group database comprising information relating to a plurality of group accounts and wherein the information comprises a group profile; and a processor configured to: a) receive, from a first wearable device, a first heartbeat identifier for a first user; b) receive, from a second wearable device, a second heartbeat identifier for a second user; c) combine the first and second heartbeat identifiers into a group identifier; d) compare the group identifier with the group profiles stored in the group database; and e) if the group identifier is determined to match one of the group profiles for one of the group accounts, authorise a group payment from the one group account.

### CROSS REFERENCE TO RELATED APPLICATIONS

This application claims priority to Singapore Application Serial No. 10201706487S, filed Aug. 8, 2017, which is incorporated herein by reference in its entirety

### FIELD OF THE INVENTION

The present invention relates to an electronic system and method for making group payments.

### BACKGROUND OF THE INVENTION

It may be desirable to share expenses between a group of people living together, travelling together, eating together or partaking in a group activity together. It may also be desirable for a group of family members or friends to share gift expenses for a common friend or family member.

There are two principal ways of sharing expenses for a group of people. Using a post-pay model, one or more persons will bear all of the expenses when they are incurred and later they will split the expenses equally between all members of the group. Thus, in this model, settlement of money between the group members only happens after an initial payment. Splitwise™ is an example of an application (“App”) which uses a post-pay model. Alternatively, using a pre-pay model, everyone pays in advance to one person in the group and that person then pays all of the joint expenses. An advantage of this model is that there is no need for later account settlement. Once all expenses are paid, any remaining balance can be divided equally between all members of the group.

In 2013, a Canadian Firm called Bionym created a wearable wristband known as Nymi™, which uses an embedded electrocardiogram (ECG) sensor to recognize the unique cardiac rhythm of users. This ECG sensor is able to match the user's ECG against a stored profile in order to authenticate the user's identity. More recently, Bionym partnered with MasterCard and the Toronto-Dominion Bank (which, together with its subsidiaries is known as TD Bank Group) to launch a pilot of a biometric payment system using the Nymi™ wristband linked to a MasterCard credit card for payments. A near-field communication (NFC) chip inside the wristband enables wireless communication with payment terminals, while the ECG sensor authenticates the user. More details of the pilot program are available via the following internet address: http://venturebeat.com/2015/08/12/mastercard-and-nymi-say-theyve-completed-the-first-heartbeat-authenticated-mobile-payment-in-the-wild/. In addition, Bionym's WO2015/011552 describes the operation of the wearable device and possible payment applications for a single user. However, there is no consideration of group payments.

Similarly, WO2012/151680 (Agrafioti et al.) describes a biometric security system which may be operable for instantaneous or continuous identity recognition. A machine learning facility is used to process physiological signals to determine the variability of each one and to identify or verify an individual from said signals. However, there is no consideration of group payments.

In CN103093264 (Qingdao Qunheng Bioscience) a portable biological feature identification storage device is disclosed. While debit cards and credit cards are considered there is no consideration of group payments.

GB2516660 (Barclays Bank) also discloses a payment authorisation system. However, in this case, the relationships between customers are used to identify a level of authorisation required for a payment. For example, where it is detected that a transaction by a first customer is related to a transaction by other customers, having a relationship with the first customer, a lower level of security is permitted.

WO2006/0262893 (HSBC) discloses a biometric identification system for facilitating a transaction in a point of sale environment. The system facilitates creating or updating a biometric template based on samples received from an existing customer belonging to an existing predetermined customer group, and approving a transaction at a point of sale terminal based on a validated sample received from a verified customer.

US2007/0198287 (Outwater) describes a system whereby an account holder may submit biometric data and receive an identification token associated with the holder's account and biometric data such that subsequent presentation of the token for access or transaction automatically triggers a verification of the biometric data. Also, disclosed is a system for securely enrolling a member of a first group into a second group.

Thus, none of the above prior art systems adequately address the desire for making a secure group payment.

Consequently, there is a need for an electronic system and method for making group payments.

### SUMMARY OF THE INVENTION

In accordance with a first aspect of the present invention there is provided an electronic system for authorising a group payment from a group account comprising:
a group database comprising information relating to a plurality of group accounts, the information comprising a group profile; and
a processor configured to:
a) receive, from a first wearable device, a first heartbeat identifier for a first user;
b) receive, from a second wearable device, a second heartbeat identifier for a second user;
c) combine the first and second heartbeat identifiers into a group identifier;
d) compare the group identifier with the group profiles stored in the group database; and
e) if the group identifier is determined to match one of the group profiles for one of the group accounts, authorise a group payment from the one group account.

Embodiments of the invention therefore provide an electronic system that can use the heartbeat of a group of people to authorise group payments.

Everybody has a unique heartbeat based on the size and shape of the person's heart, the orientation of heart valves and other physiology. Importantly, a heartbeat does not change unless the heart experiences a major cardiac event like a heart attack.

It is known to use heartbeats from individuals to authenticate a user's identity. However, the combining of two or more heartbeat signals to identify a group and authorise group payments is new. Moreover, embodiments of the invention provide for an improved group payment process whereby two or more group members can authorise payment from a group account using their heartbeats as unique passwords.

The group accounts may comprise joint accounts (i.e. current or savings accounts) and/or group wallets which are pre-loaded with funds from each group member. Thus, embodiments of the invention may provide a pre-pay solution for group expenses. Furthermore, aspects of the invention may be carried out by a group wallet application and/or a group wallet server.

The processor may be comprised in a payment terminal or a group account server (e.g. a group wallet server or joint account server).

In accordance with a second aspect of the present invention there is provided a computer-implemented method for authorising a group payment from a group account comprising:
a) storing, in a group database, information relating to a plurality of group accounts, the information comprising a group profile;
b) receiving, from a first wearable device, a first heartbeat identifier for a first user;
c) receiving, from a second wearable device, a second heartbeat identifier for a second user;
d) combining the first and second heartbeat identifiers into a group identifier;
e) comparing the group identifier with the group profiles stored in the group database; and
f) if the group identifier is determined to match one of the group profiles for one of the group accounts, authorising a group payment from the one group account.

The first and/or second heartbeat identifiers may comprise an electrocardiogram (ECG) signal or a personal code (e.g. password or personal identification number PIN) associated with an electrocardiogram (ECG) signal. The personal code may be generated or recorded during a user registration process.

The group identifier may therefore be obtained by combining ECG signals or personal codes. Accordingly, the group identifier may comprise a group ECG signal or a group code.

When the first and second heartbeat identifiers each comprise ECG signals, the step of combining the first and second heartbeat identifiers into a group identifier may comprise:
i) obtaining a first Fourier Transform using the first ECG signal;
ii) obtaining a second Fourier Transform using the second ECG signal;
iii) combining the first and second Fourier Transforms into a group Fourier Transform; and
iv) generating the group identifier by performing an Inverse Fourier Transform on the group Fourier Transform.

The method may further comprise receiving, from a third or further wearable device, a third or further heartbeat identifier for a third or further user and combining the third or further heartbeat identifier with the first and second heartbeat identifiers into the group identifier. Thus, embodiments of the invention may permit or require any number of users to provide heartbeat identifiers for the purpose of generating the group identifier.

The method may further comprise the steps of:
a) registering at least a first and a second user's heartbeat identifier with a group account; and
b) storing at least one withdrawal rule establishing criteria for authorising a group payment from the group account.

The withdrawal rule may specify that one or more of the following users' heartbeat identifiers are required in order to authorise a group payment from the group account:
ALL (wherein all users are required to authorise a group payment);
ANY TWO (wherein any two users are required to authorise a group payment);
ANY THREE (wherein any three users are required to authorise a group payment);
ANY predetermined number (wherein any predetermined number of users are required to authorise a group payment);
GROUP LEADER (wherein at least one user is designated as a group leader and the group leader alone is permitted to authorise a group payment).

The group leader may, by default, be a user who creates the group account. Individual users may be associated with a group account using his/her respective mobile number. An activation request may be sent to each mobile number to prompt each user to confirm whether they wish to join the group. If a user wishes to join the group, they will be required to register their heartbeat identifier by, for example, using a wearable device to obtain their heartbeat signal and transmitting this via their mobile device to the group account server.

Once a group account is created all members of the group may transfer money to the group account.

Each group profile may comprise a reference signal or reference code derived according to the stored withdrawal rules for each respective group account.

The withdrawal rules may be changed by updating the group profile. In some embodiments, the existing withdrawal rule may be applied in order to authorise a change to the withdrawal rule. For example, if the existing withdrawal rule is ANY TWO, two members of the group will be required to authorise a change to the withdrawal rule. In other embodiments, all members of the group may be required to authorise a change to the withdrawal rules.

The group account information may further comprise user information for each group member. For example, a name, address, mobile number, email address, reference heartbeat identifier and an amount paid by the respective user into the group account.

When a group payment is to be made, all of the required users (i.e. according to the withdrawal rule set) may be required to submit their heartbeat identifiers to the processor (i.e. of the group payment server) within a specified time period in order for the group payment to proceed. The time period may be, for example, 1 minute, 2 minutes, 3 minutes, 5 minutes, 10 minutes, 15 minutes, 30 minutes, 1 hour or 1 day. Different time periods may be set for different types of payment. For example, for a point-of-sale payment at a brick and mortar merchant a short time period may be set (e.g. 3 minutes) but for an online payment a longer time period may be set (e.g. 1 hour).

It should be noted that the group members required to authorise a group payment in accordance with the withdrawal rules need not be physically together or at the location where the payment is being made. Instead, all that is required is that the required users submit their respective heartbeat identifiers within the specified time period.

In accordance with a third aspect of the present invention there is provided a computerised network of devices for authorising a group payment from a group account comprising:

a) a first wearable device for obtaining a heartbeat identifier of a first user;
b) a second wearable device for obtaining an heartbeat identifier of a second user; and
c) the electronic system according to the first aspect of the invention.

The first and/or second wearable may comprise a heartbeat (e.g. electrocardiogram ECG) sensor and a communication device. The communication device may be configured, for example, for near-field communication (NFC), Wifi, Bluetooth, Lifi, infrared or radio-frequency (RF) communication.

Further wearable devices may be required for obtaining a heartbeat identifier of further users.

In some embodiments, the wearable devices may be configured to communicate directly with the electronic system and in other embodiments, the wearable devices may be configured to communicate with the electronic system via a user's mobile device (e.g. smartphone, tablet, laptop or computer).

As used throughout this specification, the term payment card may comprise any suitable cashless payment mechanism, such as a credit card, a debit card, a prepaid card, a charge card, a membership card, a promotional card, a frequent flyer card, an identification card, a gift card, and/or any other physical or electronic device that may hold payment account information, such as digital wallets.

Embodiments of the invention may be expressed as a network of communicating devices (i.e. a “computerized network”). It may further be expressed in terms of a software application downloadable into a computer device to facilitate the method. The software application may be a computer program product, which may be stored on a non-transitory computer-readable medium on a tangible data-storage device (such as a storage device of a server, or one within a user device).

### BRIEF DESCRIPTION OF THE DRAWINGS

Embodiments of the invention will now be described by way of example only with reference to the following drawings, in which:

FIG. 1 illustrates a computer-implemented method for authorising a group payment from a group account in accordance with a first embodiment of the invention;

FIG. 2 illustrates a computerised network of electronic devices for performing the method of FIG. 1; and

FIG. 3 illustrates the combining of two user's heartbeats into a group heartbeat signal in accordance with an embodiment of the invention;

FIG. 4 illustrates performing a Fourier Transform on a user's heartbeat signal to convert it into a series of sine and cosine waves in accordance with an embodiment of the invention;

FIG. 5 illustrates a method for registering a user's heartbeat in accordance with an embodiment of the invention;

FIG. 6 illustrates a method for creating a group account in accordance with an embodiment of the invention;

FIG. 7 illustrates a method for setting a withdrawal rule for a group account in accordance with an embodiment of the invention;

FIG. 8 illustrates a method for authorising a group payment from a group account in accordance with an embodiment of the invention;

FIG. 9 illustrates functional modules within a server of FIG. 2;

FIG. 10 illustrates a block diagram of the technical architecture of a server of FIG. 2; and

FIG. 11 illustrates a block diagram of the technical architecture of a mobile device of FIG. 2.

### DETAILED DESCRIPTION OF CERTAIN EMBODIMENTS

FIG. 1 shows a computer-implemented method 10 for authorising a group payment from a group account in accordance with a first embodiment of the invention. The method 10 comprises the following steps:
a) storing, in a group database, information relating to a plurality of group accounts, the information comprising a group profile;
b) receiving, from a first wearable device, a first heartbeat identifier for a first user;
c) receiving, from a second wearable device, a second heartbeat identifier for a second user;
d) combining the first and second heartbeat identifiers into a group identifier;
e) comparing the group identifier with the group profiles stored in the group database; and
f) if the group identifier is determined to match one of the group profiles for one of the group accounts, authorising a group payment from the one group account.

If the group identifier is determined not to match one of the group profiles, the group payment is not authorised and a request may be made for the first and second heartbeat identifiers to be re-sent and the method above to be repeated.

In some embodiments a third or further heartbeat identifier for a third or further user may be received from a third or further wearable device. The third or further heartbeat identifier may be combined with the first and second heartbeat identifiers into the group identifier. Thus, embodiments of the invention may permit or require any number of users to provide heartbeat identifiers for the purpose of generating the group identifier via which the group payment is authorised.

The group accounts themselves may comprise joint accounts (i.e. current or savings accounts) and/or group wallets which are pre-loaded with funds from each group member.

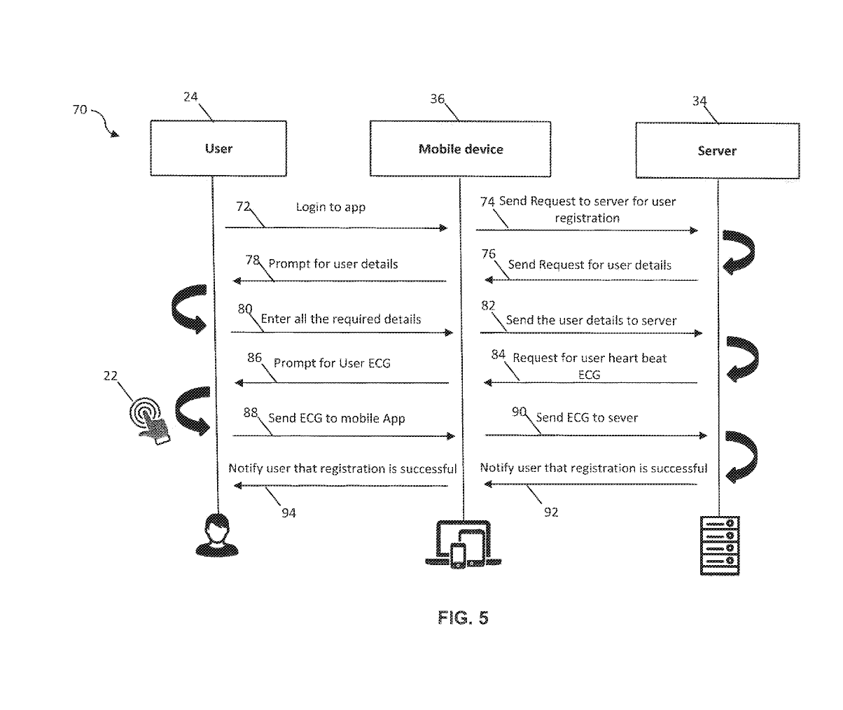

As will be explained in more detail below with reference to FIG. 5 each user will be required to register a heartbeat identifier with the group account and the group profiles will be derived from prescribed combinations of the heartbeat identifiers for each group member.

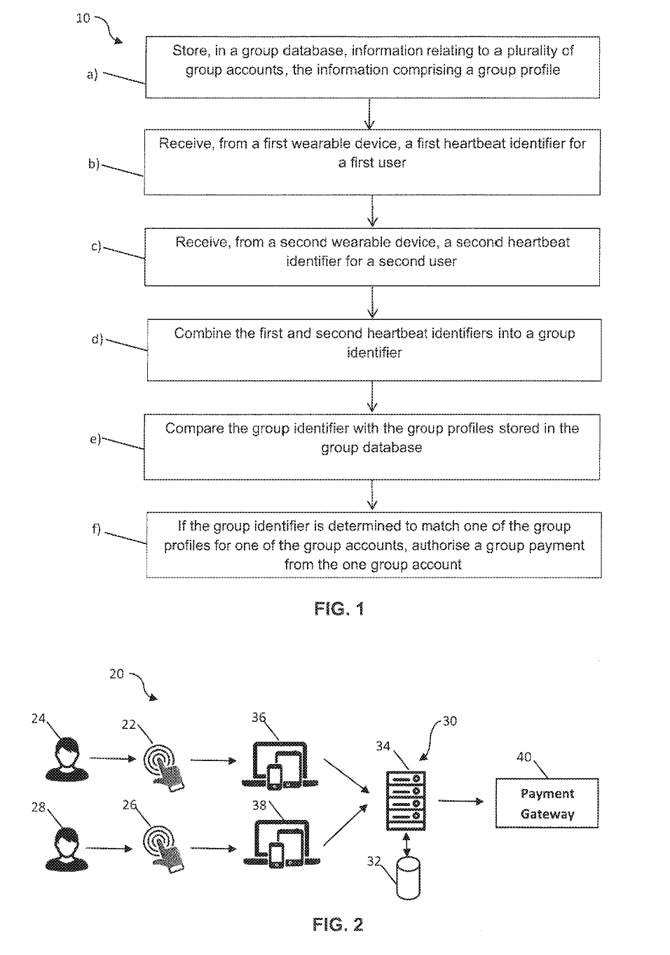

FIG. 2 illustrates a computerized network 20 of electronic devices for performing the method of FIG. 1. Thus, the network 20 comprises a first wearable device 22 for obtaining a heartbeat identifier of a first user 24, a second wearable device 26 for obtaining a heartbeat identifier of a second user 28 and an electronic system 30 for authorising a group payment from a group account. The electronic system 30 comprises a group database 32 comprising information relating to a plurality of group accounts, the information comprising a group profile; and a processor, which in this case is comprised in a server 34. The processor is configured to carry out the steps detailed above and as shown in FIG. 1.

Further wearable devices may be required for obtaining heartbeat identifiers for one or more further users.

In a particular embodiment, each wearable device 22, 26 comprises a heartbeat electrocardiogram (ECG) sensor and a communication device configured for near-field communication (NFC) with a respective user's mobile device 36, 38. In turn, each mobile device 36, 38 is configured for communication with the server 34 (e.g. via SMS, 3G or 4G communication protocols) and the server 34 itself is configured for communication with a payment gateway 40 for processing of a group payment if and when it is authorised in accordance with embodiments of the invention.

In other embodiments, the wearable devices 22, 26 may be configured to communicate directly with the server 34 (i.e. by-passing the mobile devices 36, 38).

In some embodiments, the server 34 may be incorporated into a payment terminal (i.e. Point of Sale POS) or accessible via a POS. In some embodiments, the server 34 may be constituted by a group wallet application server.

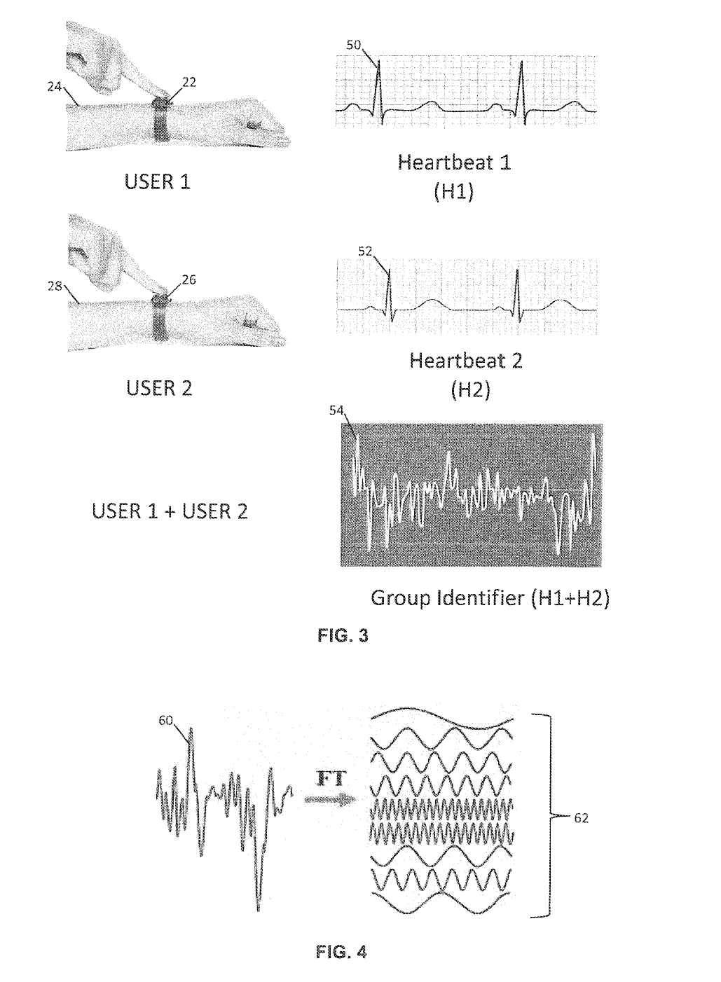

FIG. 3 illustrates the combining of two user's heartbeats into a group identifier in the form of a group heartbeat signal in accordance with an embodiment of the invention. The first user 24 (User 1) is wearing the first wearable device 22 around one of his/her wrists, which is configured to measure the first user's heartbeat and to record a periodic electrocardiogram (ECG) signal 50 (referred to as Heartbeat 1 or H1). The second user 28 (User 2) is wearing the second wearable device 26 around one of his/her wrists, which is configured to measure the second user's heartbeat and to record a periodic electrocardiogram (ECG) signal 52 (referred to as Heartbeat 2 or H2). It will be noted that each of the heartbeat signals H1 and H2 are unique to a respective one of the users.

Each wearable device 22, 26 transmits the recorded heartbeat signal H1 or H2 via the respective user's mobile device 36, 38 to the server 34. Upon receipt of H1 and H2, the server 34 combines H1 and H2 by adding H1 and H2 together to form a group identifier in the form of a periodic group heartbeat signal 54. More specifically, H1 and H2 are each broken down into a series of sine and cosine waves by application of a Fourier Transform (FT). FIG. 4 illustrates performing a Fourier Transform on a signal 60 to convert it into a series 62 of sine and cosine waves representing the original signal 60. Once each of the heartbeat signals H1 and H2 have been transformed into a series of sine and cosine waves, all of the resulting waves are added together and an Inverse Fourier Transform is performed to convert the combined waves into a single unique periodic group heartbeat signal 54 constituting a group identifier, which can be used as a group password. In other embodiments, H1 and H2 may be combined in a different way to form the group identifier.

It will be understood that each wearable device is effectively employed to authenticate an individual user or member of a group and by combining the unique heartbeat signals from two or more users into a group identifier it is possible to authenticate the identity of the group.

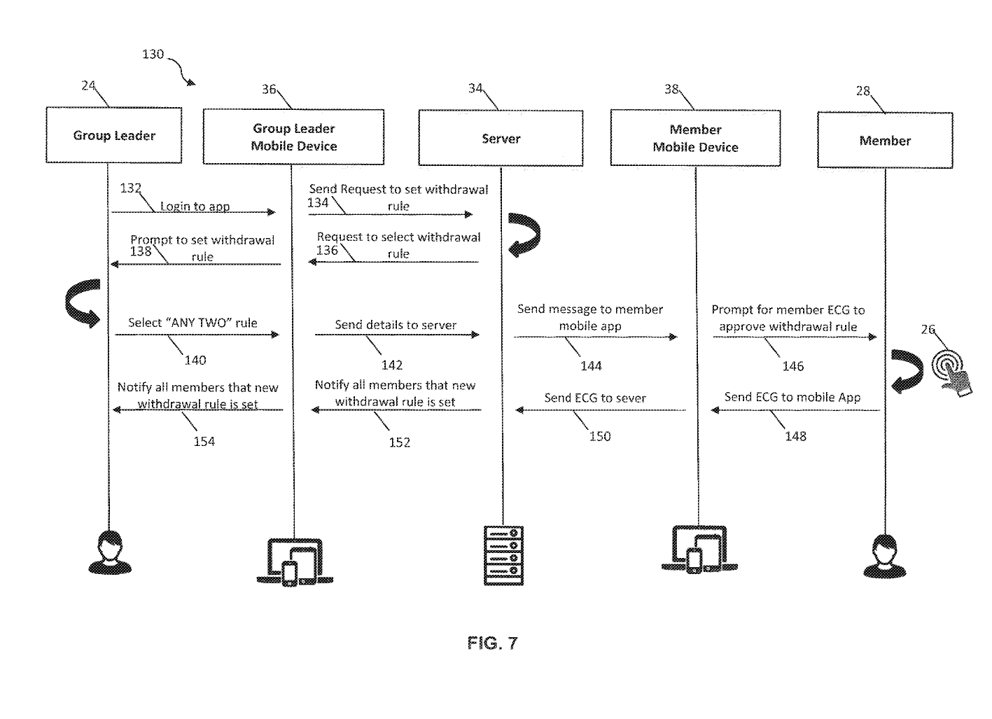

Once the group identifier is obtained it is matched against stored group profiles in the group database 32. In this embodiment, the group profiles comprise reference signals derived according to stored withdrawal rules for each respective group account. The withdrawal rules will be established during an initial registration or group account set-up procedure and may be subsequently modified as described below in relation to FIG. 7. The withdrawal rules may specify that one or more of the following users' heartbeat identifiers (i.e. H1 and H2) are required in order to authorise a group payment from the group account:

ALL (wherein all users are required to authorise a group payment);
ANY TWO (wherein any two users are required to authorise a group payment);
ANY THREE (wherein any three users are required to authorise a group payment);
ANY predetermined number (wherein any predetermined number of users are required to authorise a group payment);
GROUP LEADER (wherein at least one user is designated as a group leader and the group leader alone is permitted to authorise a group payment).

It should be noted that the above rules are examples only and many variations are possible. For example, the group leader's heartbeat may always be required. Moreover, any form of rules may be provided to support various rights management and authentication requirements relating to the use of heartbeat identifiers for a group payment in accordance with embodiments of the invention.

As an example, in this particular embodiment, the withdrawal rule is set to ANY TWO such that the heartbeat signals (H1 and H2) from any two members in the group are required to authorise a group payment. Accordingly, the group profile for the group account comprises a series of reference signals each of which is derived from a combination of two different member's registered heartbeat signals. As such, whenever any two group member's heartbeat signals are combined into a group identifier, the group identifier will match with one of the group profiles for the group account thereby confirming that the group payment is authorised to proceed.

It is noted that any periodic signal (such as a heartbeat) can be approximated by a sum of many sinusoids at harmonic frequencies of the signal, with appropriate amplitude and phase, using a Fourier Transform. In an example, gₚ(t) represents a periodic signal with period of Tₒ. Using a Fourier Transform it is possible to resolve the signal of gₚ(t) into an infinite sum of sine and cosine terms. More specifically, after a Fourier series expansion of gₚ(t), the form is in accordance with Equation (1) where the terms aₙ and bₙ are unknown amplitudes of the cosine and sine terms.

$${g}_{p} ( t ) = {a}_{o} + 2 {\sum }_{1}^{∞} [ ( {a}_{n} * cos ( 2 * π * n * t / {T}_{o} ) + ( {b}_{n} * sin ( 2 * π * n * t / {T}_{o} ) ] \qquad Equation ( 1 )$$

In some embodiments, each user's ECG signal 50, 52 may be mapped to a personal code (e.g. password or personal identification number PIN) generated by the server 34 at the time the user register his/her heartbeat. The personal code will therefore constitute the heartbeat identifier for each user and it may be kept secure and unknown to anyone except the server 34. Accordingly, when a group payment is to be authorised, the server 34 will receive the ECG signals 50, 52 from the required users and will determine the personal codes associated with each of the ECG signals 50, 52. The personal codes will be then be combined into the group identifier (e.g. by addition or another pre-defined process). As above, the group identifier, which in this case is a group code as opposed to a group signal, will be matched against the stored group profiles according to the set withdrawal rules to determine whether the group payment is authorised. Notably, the group profiles in this case will also be group codes derived from the personal codes associated with each user's ECG.

FIG. 5 shows a method 70 for registering a user's heartbeat in accordance with an embodiment of the invention. In a first step 72, the user 24 logs in to a group payment application “app” on his/her mobile device 36. In a step 74, the user instructs the mobile device 36 to send a request for user registration to the server 34, the request comprises a number for the user's mobile device 36. In a step 76, the server 34 checks whether the number exists in a user database 32 and, if not, returns a request to the mobile device 36 for user details and in a step 78 the mobile device 36 prompts the user 24 to enter his/her user details. In a step 80, the user 24 enters all of the required details (e.g. name, mobile number, address, email address) into his/her mobile device 36 and in a step 82, the mobile device 36 sends the user details to the server 34. In a step 84, the server 34 saves the user details in the user database 32 and sends a request for the user's heartbeat ECG to the mobile device 36. In a step 86, the mobile device 36 prompt the user to provide his/her heartbeat ECG. In a step 88, the user 24 activates his/her wearable device 22 to record his/her ECG and transmit the ECG signal (digitally encoded) via Bluetooth or NFC to the mobile device 36. In a step 90, the mobile device sends the ECG signal to the server 34. The server 34 stores the ECG signal in the user database 32 and notifies the mobile device that the registration is successful in a step 92. Lastly, in a step 94, the mobile device 36 communicates to the user 24 that the registration is successful.

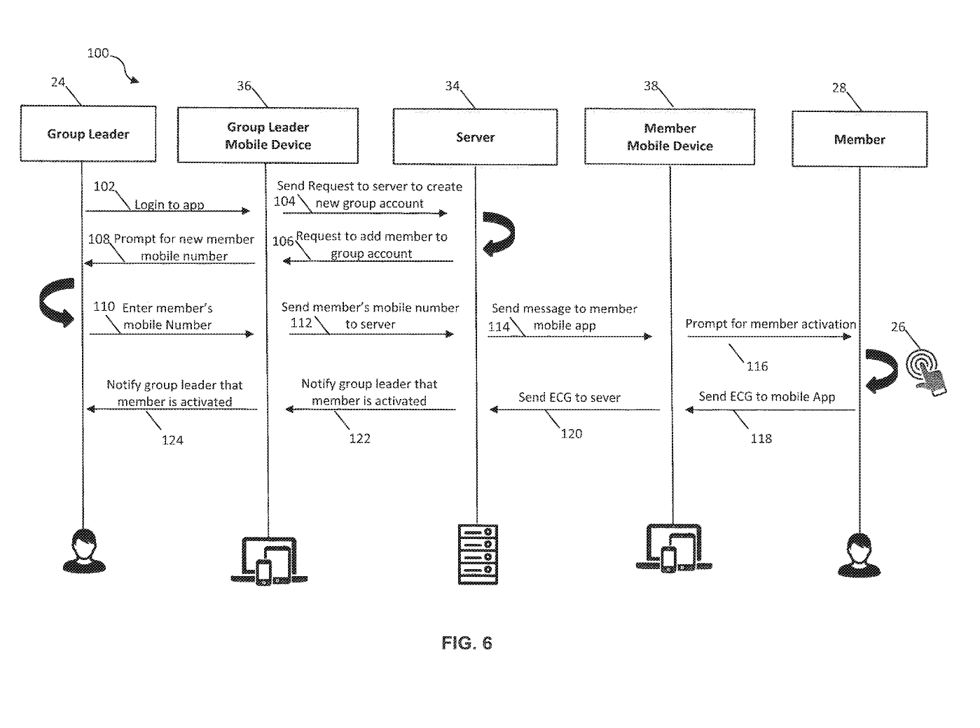

Once a user 24 is registered in accordance with method 70, that user 24 may take the role of a group leader or first user and may create a group account in accordance with the method 100 shown in FIG. 6. In a first step 102, the group leader logs in to the group payment application “app” on his/her mobile device 36. In a step 104, the group leader instructs the mobile device 36 to send a request to the server 34 to create a new group account. In a step 106, the server 34 returns a request to add a member to the group account to the mobile device 36 of the group leader and in a step 108 the mobile device 36 prompts the group leader to enter a mobile number for a new member. In a step 110, the group leader enters the mobile number for the new member into his/her mobile device 36 and in a step 112, the mobile device 36 sends the member's mobile number to the server 34. In a step 114, the server 34 checks whether the member's mobile number matches a number in the user database 32 and, if so, sends an activation message to the member's mobile device 38. The member's mobile device 38 then prompts the member 28 to provide his/her ECG in order to join the group account in a step 116. In a step 118, the member 28 activates his/her wearable device 26 to record his/her ECG and transmit the ECG signal (digitally encoded) via Bluetooth or NFC to the member's mobile device 38. In a step 120, the member's mobile device 38 sends the ECG signal to the server 34. The server 34 verifies the ECG signal against the member's ECG signal already stored in the user database 32 and, if it matches, notifies the group leader's mobile device 36 (and, optionally, the mobile devices of all other members of the group) that the new member has been successfully added to the group account, in a step 122. Lastly, in a step 124, the group leader's mobile device 36 (and, optionally, the mobile devices of all other members of the group) communicates to the group leader (and other members) that the new member has been successfully added to the group account.

If any new members are not already registered with the server 34 they will be prompted to register in accordance with method 70 before they can be added to the group account.

Once all members are successfully added to the group account the group leader will be prompted to select the withdrawal rules and the server 34 will notify these to all of the group members. In some embodiments, the group members may be required to accept the withdrawal rules and/or any changes to them by submitting their heartbeat ECGs. Accordingly, FIG. 7 illustrates a method 130 for setting a withdrawal rule for a group account in accordance with an embodiment of the invention.

In a first step 132, the group leader logs in to the group payment application “app” on his/her mobile device 36. In a step 134, the group leader instructs the mobile device 36 to send a request to the server 34 to set a withdrawal rule. In a step 136, the server 34 returns a request to select a withdrawal rule to the mobile device 36 of the group leader and in a step 138 the mobile device 36 prompts the group leader to set a withdrawal rule. In a step 140, the group leader enters the withdrawal rule (in this case, ANY TWO—wherein any two members are required to authorise a group payment) into his/her mobile device 36 and in a step 142, the mobile device 36 sends the withdrawal rule to the server 34. In a step 144, the server 34 sends a message to each group member's mobile device 38. Each member's mobile device 38 then prompts the member 28 to provide his/her ECG in order to approve the withdrawal rule in a step 146. In a step 148, the member 28 activates his/her wearable device 26 to record his/her ECG and transmit the ECG signal (digitally encoded) via Bluetooth or NFC to the member's mobile device 38. In a step 150, the member's mobile device 38 sends the ECG signal to the server 34. The server 34 verifies the ECG signal provided by each member against each member's ECG signal already stored in the user database 32 and, if each one matches, activates the withdrawal rule, generates all permitted permutations and combinations of the member's ECG signals (in accordance with the withdrawal rule), stores the resulting group signals in the group profile and notifies the group leader's mobile device 36 (and, optionally, the mobile devices of all other members of the group) that the withdrawal rule has been successfully set, in a step 152. Lastly, in a step 154, the group leader's mobile device 36 (and, optionally, the mobile devices of all other members of the group) communicates to the group leader (and other members) that the withdrawal rule has been successfully set for the group account.

In some embodiments, the group leader may be permitted to modify the withdrawal rules without requiring the approval of the other group members.

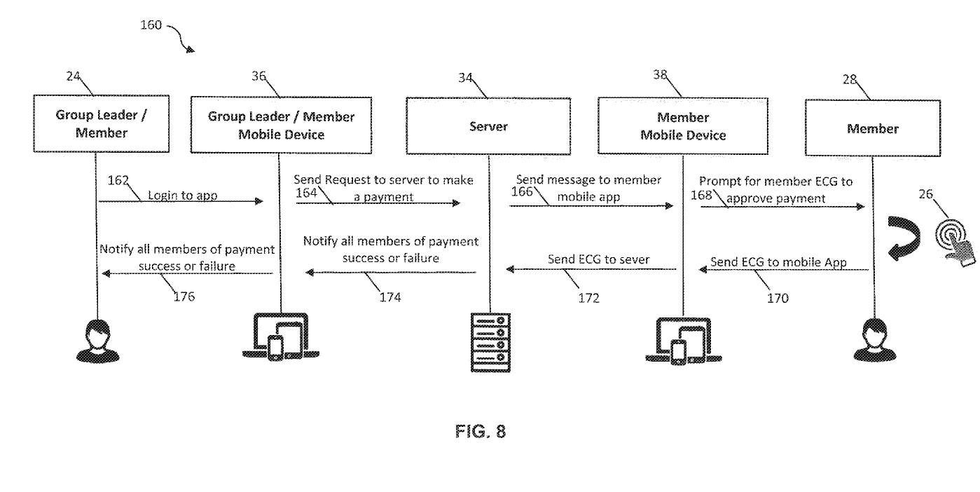

FIG. 8 illustrates a method 160 for authorising a group payment from a group account in accordance with an embodiment of the invention. Depending on the set withdrawal rules any group member 24 may be permitted to initiate a group payment by logging into the group payment application “app” on his/her mobile device 36 in a first step 162. In a step 164, the member 24 instructs the mobile device 36 to send a request to the server 34 to make a group payment, the request will include payment details comprising the payment amount and receiver's account or wallet details (e.g. receiver's mobile number). In a step 166, the server 34 checks the withdrawal rule, sends a message to each group member's mobile device 38 that may be required to authorise the group payment and starts a timer (e.g. to timeout after 15 minutes). For example, if the withdrawal rule is ANY TWO, the server 34 will message all members of the group so that any two of the members can authorise the group payment. Each member's mobile device 38 then prompts the member 28 to provide his/her ECG in order to approve the group payment in a step 168. In a step 170, the member 28 activates his/her wearable device 26 to record his/her ECG and transmit the ECG signal (digitally encoded) via Bluetooth or NFC to the member's mobile device 38. In a step 172, the member's mobile device 38 sends the ECG signal to the server 34. If the required number or members in accordance with the withdrawal rule have provided their ECG signals to the server 34 before time runs out on the timer, the server 34 will combine the required member ECG signals into the group identifier and compare this with the stored group profiles to determine whether there is a match and the group payment is authorised. If the group payment is authorised, the server 34 will initiate the group payment in accordance with the payment details. Thus, a payment for the payment amount will be made from the group account (e.g. wallet) to the receiver's account (e.g. wallet). Furthermore, the server 34 will notify the mobile devices 36 of all members of the group as to whether the payment was successful or failed, in a step 174. Lastly, in a step 176, the mobile devices 36 of all members of the group will communicate to each member whether the payment has been successful or failed.

Notably, one or more of the members required to authorise a group payment in accordance with the withdrawal rules need to not be physically present when the group payment is initiated since they will be notified though their mobile devices to provide their heartbeat ECG signal for authorisation purposes and this may be performed remotely.

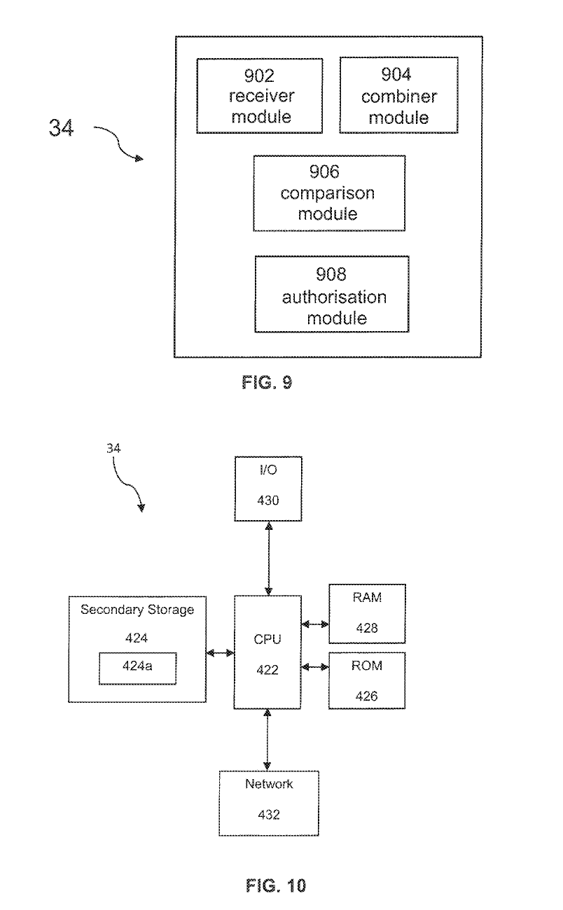

FIG. 9 illustrates functional modules within the server 34 shown in FIG. 2. In this case, the server 34 comprises a receiver module 902, a combiner module 904, a comparison module 906 and an authorisation module 908. The receiver module 902 is configured to receive, from a first wearable device, a first heartbeat identifier for a first user, and to receive, from at least a second wearable device, at least a second heartbeat identifier for at least a second user. The combiner module 904 is configured to combine the first and second heartbeat identifiers into a group identifier. The comparison module 906 is configured to compare the group identifier with the group profiles stored in the group database and if the group identifier is determined to match one of the group profiles for one of the group accounts, the authorisation module 908 is configured to authorise a group payment from the group account.

FIG. 10 is a block diagram showing a technical architecture of the server 34.

The technical architecture includes a processor 422 (which may be referred to as a central processor unit or CPU) that is in communication with memory devices including secondary storage 424 (such as disk drives), read only memory (ROM) 426, random access memory (RAM) 428. The processor 422 may be implemented as one or more CPU chips. The technical architecture may further comprise input/output (I/O) devices 430, and network connectivity devices 432.

The secondary storage 424 is typically comprised of one or more disk drives or tape drives and is used for non-volatile storage of data and as an over-flow data storage device if RAM 428 is not large enough to hold all working data. Secondary storage 424 may be used to store programs which are loaded into RAM 428 when such programs are selected for execution.

In this embodiment, the secondary storage 424 has a processing component 424a comprising non-transitory instructions operative by the processor 422 to perform various operations of the method of the present disclosure. The ROM 426 is used to store instructions and perhaps data which are read during program execution. The secondary storage 424, the RAM 428, and/or the ROM 426 may be referred to in some contexts as computer readable storage media and/or non-transitory computer readable media.

I/O devices 430 may include printers, video monitors, liquid crystal displays (LCDs), plasma displays, touch screen displays, keyboards, keypads, switches, dials, mice, track balls, voice recognizers, card readers, paper tape readers, or other well-known input devices.

The network connectivity devices 432 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identity (RFID), and/or other air interface protocol radio transceiver cards, and other well-known network devices. These network connectivity devices 432 may enable the processor 422 to communicate with the Internet or one or more intranets. With such a network connection, it is contemplated that the processor 422 might receive information from the network, or might output information to the network in the course of performing the above-described method operations. Such information, which is often represented as a sequence of instructions to be executed using processor 422, may be received from and outputted to the network, for example, in the form of a computer data signal embodied in a carrier wave.

The processor 422 executes instructions, codes, computer programs, scripts which it accesses from hard disk, floppy disk, optical disk (these various disk based systems may all be considered secondary storage 424), flash drive, ROM 426, RAM 428, or the network connectivity devices 432. While only one processor 422 is shown, multiple processors may be present. Thus, while instructions may be discussed as executed by a processor, the instructions may be executed simultaneously, serially, or otherwise executed by one or multiple processors.

Although the technical architecture is described with reference to a computer, it should be appreciated that the technical architecture may be formed by two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the technical architecture 420 to provide the functionality of a number of servers that is not directly bound to the number of computers in the technical architecture 420. In an embodiment, the functionality disclosed above may be provided by executing the application and/or applications in a cloud computing environment. Cloud computing may comprise providing computing services via a network connection using dynamically scalable computing resources. A cloud computing environment may be established by an enterprise and/or may be hired on an as-needed basis from a third party provider.

It is understood that by programming and/or loading executable instructions onto the technical architecture, at least one of the CPU 422, the RAM 428, and the ROM 426 are changed, transforming the technical architecture in part into a specific purpose machine or apparatus having the novel functionality taught by the present disclosure. It is fundamental to the electrical engineering and software engineering arts that functionality that can be implemented by loading executable software into a computer can be converted to a hardware implementation by well-known design rules.

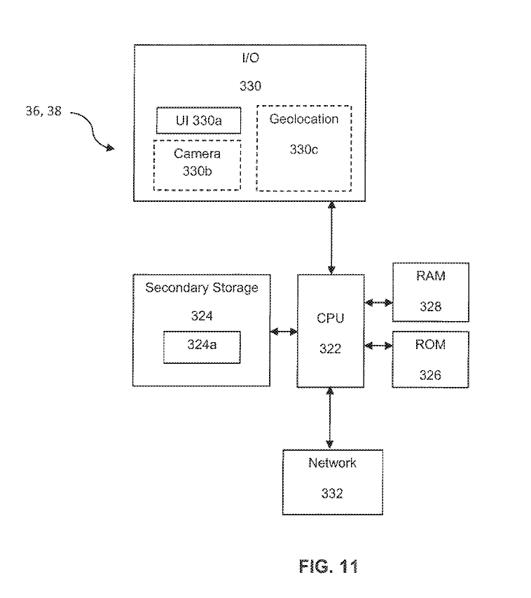

FIG. 11 is a block diagram showing a technical architecture of the user's mobile devices 36, 38.

The technical architecture includes a processor 322 (which may be referred to as a central processor unit or CPU) that is in communication with memory devices including secondary storage 324 (such as disk drives or memory cards), read only memory (ROM) 326, random access memory (RAM) 328. The processor 322 may be implemented as one or more CPU chips. The technical architecture further comprises input/output (I/O) devices 330, and network connectivity devices 332.

The I/O devices comprise a user interface (UI) 330a. In the case of the customer mobile device 28, a camera 330b and a geolocation module 330c may also be provided. The UI 330a may comprise a touch screen, keyboard, keypad or other known input device. The camera 330b allows a user to capture images and save the captured images in electronic form. The geolocation module 330c is operable to determine the geolocation of the communication device using signals from, for example global positioning system (GPS) satellites.

The secondary storage 324 is typically comprised of a memory card or other storage device and is used for non-volatile storage of data and as an over-flow data storage device if RAM 328 is not large enough to hold all working data. Secondary storage 324 may be used to store programs which are loaded into RAM 328 when such programs are selected for execution.

In this embodiment, the secondary storage 324 has a processing component 324a, comprising non-transitory instructions operative by the processor 322 to perform various operations of the method of the present disclosure. The ROM 326 is used to store instructions and perhaps data which are read during program execution. The secondary storage 324, the RAM 328, and/or the ROM 326 may be referred to in some contexts as computer readable storage media and/or non-transitory computer readable media.

The network connectivity devices 332 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identity (RFID), and/or other air interface protocol radio transceiver cards, and other well-known network devices. These network connectivity devices 332 may enable the processor 322 to communicate with the Internet or one or more intranets. With such a network connection, it is contemplated that the processor 322 might receive information from the network, or might output information to the network in the course of performing the above-described method operations. Such information, which is often represented as a sequence of instructions to be executed using processor 322, may be received from and outputted to the network, for example, in the form of a computer data signal embodied in a carrier wave.

The processor 322 executes instructions, codes, computer programs, scripts which it accesses from hard disk, floppy disk, optical disk (these various disk based systems may all be considered secondary storage 324), flash drive, ROM 326, RAM 328, or the network connectivity devices 332. While only one processor 322 is shown, multiple processors may be present. Thus, while instructions may be discussed as executed by a processor, the instructions may be executed simultaneously, serially, or otherwise executed by one or multiple processors.

Whilst the foregoing description has described exemplary embodiments, it will be understood by those skilled in the art that many variations of the embodiments can be made in accordance with the appended claims.

1. An electronic system for authorising a group payment from a group account comprising:
a) a group database comprising information relating to a plurality of group accounts, the information comprising a group profile; and
b) a processor configured to:
i) receive, from a first wearable device, a first heartbeat identifier for a first user;
ii) receive, from a second wearable device, a second heartbeat identifier for a second user;
iii) combine the first and second heartbeat identifiers into a group identifier;
iv) compare the group identifier with the group profiles stored in the group database; and
v) if the group identifier is determined to match one of the group profiles for one of the group accounts, authorise a group payment from the one group account.

2. The system according to claim 1 wherein the group accounts comprise joint accounts and/or group wallets which are pre-loaded with funds from each group member.

3. The system according to claim 1 wherein the processor is comprised in a payment terminal or a group account server.

4. A computer-implemented method for authorising a group payment from a group account comprising:
a) storing, in a group database, information relating to a plurality of group accounts, the information comprising a group profile;
b) receiving, from a first wearable device, a first heartbeat identifier for a first user;
c) receiving, from a second wearable device, a second heartbeat identifier for a second user;
d) combining the first and second heartbeat identifiers into a group identifier;
e) comparing the group identifier with the group profiles stored in the group database; and
f) if the group identifier is determined to match one of the group profiles for one of the group accounts, authorising a group payment from the one group account.

5. The method according to claim 4 wherein the first and/or second heartbeat identifiers comprise an electrocardiogram (ECG) signal or a personal code associated with an electrocardiogram (ECG) signal.

6. The method according to claim 5 wherein the first and second heartbeat identifiers each comprise ECG signals and the step of combining the first and second heartbeat identifiers into a group identifier comprises:
a) obtaining a first Fourier Transform using the first ECG signal;
b) obtaining a second Fourier Transform using the second ECG signal;
c) combining the first and second Fourier Transforms into a group Fourier Transform; and
d) generating the group identifier by performing an Inverse Fourier Transform on the group Fourier Transform.

7. The method according to claim 4 further comprising receiving, from a third or further wearable device, a third or further heartbeat identifier for a third or further user and combining the third or further heartbeat identifier with the first and second heartbeat identifiers into the group identifier.

8. The method according to claim 4 further comprise the steps of:
a) registering at least a first and a second user's heartbeat identifier with a group account; and
b) storing at least one withdrawal rule establishing criteria for authorising a group payment from the group account.

9. The method according to claim 8 wherein the withdrawal rule specifies that one or more of the following users' heartbeat identifiers are required in order to authorise a group payment from the group account:
a) ALL (wherein all users are required to authorise a group payment);
b) ANY TWO (wherein any two users are required to authorise a group payment);
c) ANY THREE (wherein any three users are required to authorise a group payment);
d) ANY predetermined number (wherein any predetermined number of users are required to authorise a group payment);
e) GROUP LEADER (wherein at least one user is designated as a group leader and the group leader alone is permitted to authorise a group payment).

10. The method according to claim 9 wherein a user who creates the group account is initially designated as the group leader by default.

11. The method according to claim 4 wherein individual users are associated with a group account using his/her respective mobile number.

12. The method according to claim 11 wherein an activation request is sent to each mobile number to prompt each user to confirm whether they wish to join the group account.

13. The method according to claim 9 wherein each group profile comprises a reference signal or reference code derived according to the stored withdrawal rules for each respective group account.

14. The method according to claim 8 wherein the withdrawal rule is changed by updating the group profile.

15. The method according to claim 14 wherein the stored withdrawal rule is applied in order to authorise a change to the withdrawal rule.

16. The method according to claim 14 wherein all members of the group account are required to authorise a change to the withdrawal rule.

17. The method according to claim 4 wherein the group account information further comprises user information for each group member.

18. The method according to claim 8 wherein all of the required users according to the stored withdrawal rule must submit their heartbeat identifiers within a specified time period in order for the group payment to proceed.

19. A computerised network of devices for authorising a group payment from a group account comprising:
a) a first wearable device for obtaining a heartbeat identifier of a first user;
b) a second wearable device for obtaining an heartbeat identifier of a second user; and
c) the electronic system according to claim 1.

20. A non-transitory computer-readable medium having stored thereon program instructions for causing at least one processor to perform the method according to claim 1.
